(19) United States
(12) Patent Application Publication
(10) Pub. No.: US 2008/0275360 A1
(43) Pub. Date: Nov. 6, 2008

(54) Arrangement and Method for Assessing the Motility of a Generally Tubular Anatomical Organ

(76) Inventor: Lars Fandriks, Askim (SE)

Correspondence Address: SCHWEGMAN, LUNDBERG & WOESSNER, P.A., P.O. BOX 2938, MINNEAPOLIS, MN, 55402, US

(21) Appl. No.: 11/579,524

(22) PCT Filed: May 4, 2005
(86) PCT No.: PCT/SE2005/00656

§ 371 Date: Nov. 3, 2006

(30) Foreign Application Priority Data
May 4, 2004 (SE) 0401151-6

(51) Int. Cl. A61B 5/0488

(52) U.S. Cl. 600/546

(57) ABSTRACT

The invention relates to an arrangement for assessing the motility of a generally tubular anatomical organ (1), said arrangement comprising a longitudinally extending catheter (6) being adapted for introducing into said organ (1). The invention furthermore comprises an electrode arrangement (7) arranged along the longitudinal extension of said catheter (6), a detection unit (8) for measuring the electric potential (V(d)) at least partly along the length of said organ (1) and an evaluation unit (13) for detecting a generally step-like change (19) in the measured electric potential and determining the distance between a position along said catheter (6) corresponding to said step-like change (19) and a predetermined fixed point (16) to which the catheter (6) can be attached. The invention also relates to a method for such motility assessment.

### TECHNICAL FIELD

The present invention relates to an arrangement for assessing the motility of a generally tubular anatomical organ, said arrangement comprising a longitudinally extending catheter being adapted for introducing into said organ.

The invention also relates to a method for assessing the motility of a generally tubular anatomical organ, said method comprising introducing a longitudinally extending catheter into said organ.

### STATE OF THE ART

The esophagus is an extended hollow organ that connects the throat with the stomach via the thoracic cavity. The esophagus consists of one outer muscular layer oriented longitudinally and one inner muscular layer with its muscle fibres oriented circumferentially. The esophageal inside is covered by a mucosa with a squamous epithelium facing the lumen. The main function of the esophagus is to transport ingested food from the oral cavity into the abdominal part of the gastrointestinal system where the digestive and absorptive processes take place. It follows that the esophageal epithelium does not contribute to digestion as does the mucosal epithelium of the rest of the gut.

The distal part of the esophagus also has a valvular function to prevent gastric luminal solid and liquid contents from entering into the esophagus but allow a selective evacuation of swallowed air. This valvular function is named the “lower esophageal sphincter” (LES) which thus is a part of the distal esophagus close to the connection to the stomach. Together with external forces from the surrounding diaphragm, the LES exerts a relatively high intraluminal pressure that is released in association to swallowing or belching by complex neuro-hormonal regulation.

Diseases of the esophagus are very common and related both to muscular and/or mucosal diseases. One very common symptom related to the esophagus is non-cardiac chest pain or “heart burn”. Such symptoms are extremely common; 24% of the people in the industrialized world report heart burn on a monthly basis and a significant proportion of these consult medical services. However, although gastroesophageal reflux is sometimes obvious, there are a number of uncertainties on when and why gastroesophageal reflux causes heart-burn symptoms, particularly in cases without signs of esophageal mucosal injuries.

One well known cause of heart burn is dysfunction of the valvular property of LES allowing acidic and proteolytically erosive luminal contents of the stomach to regurgitate into the esophageal lumen. If frequent and long-standing such reflux episodes can elicit inflammatory reactions (esophagitis) of the mucosa and generate symptoms. This disorder is commonly called “gastro-esophageal-reflux disease” (GERD). The most prominent regurgitated erosive factor is the acid originating from the stomach. It follows that drugs inhibiting the gastric acidity are very successful for symptom-relief in these cases. Typically this disorder is diagnosed by 1./patients history; 2./endoscopic findings of esophagitis; and 3./acidic reflux episodes of long duration and frequency as recorded by pH electrode positioned in the esophagus over 24 hours (24 h pH-metry). However, many patients complaining for heart burn have normal 24 h pH-metry and no esophagitis. This condition in usually called “non-erosive reflux disease” (NERD) or “functional heartburn” and has an obscure pathophysiology involving for example bile-contaminated non-acid reflux or muscular dysfunctional disorders of the esophagus.

It has been suggested that esophageal symptoms, independent of if they are elicited by luminal acid or by unknown factors, are generated by longstanding contractions in the longitudinally oriented muscular layer. These sustained axial contractions of the esophagus are monitored by sensory nerves that in turn signal to the central nervous system.

As regards prior art technology, it should be noted that activity in the longitudinal muscular layer has been indirectly assessed by analysing changes of the thickness of the muscular layer as recorded by intraluminal ultrasound devices. In this regard, it can be noted that the document “Sustained esophageal contraction: a motor correlate of heartburn symptom”; N. Pehlivanov et al; Am. J. Physiol. Gastrointest Liver Physiol; 281: G743-G751, 2001, teaches a system and method for measuring the esophageal muscle thickness by means of ultrasound imaging of the esophagus. Such measurements of the esophageal muscle thickness can be used as an indication of muscle contraction. However, the system has certain drawbacks. Firstly, it should be noted that a relatively substantial effort is required for the analysis in order to fully assess the results of the measurements. In particular, it can be expected that the results must be evaluated by skilled examinators. However, pain-sensations can also be elicited by longitudinal extension of gastrointestinal tissue as for example when the esophagus is influence by shortening of the stomach and duodenum.

Furthermore, a classical way of investigating movements of the esophagus is the barium contrasted x-ray examination. However, although giving a dynamic picture of a barium swallow radiological examinations are impractical and associated with hazardous radiation. It should also be noted that both ultrasound and x-ray based methods give an on-the-spot reflection that can miss aberrations that occur over time.

More detailed analyses of longitudinal movements in the distal esophagus have been reported using radio-opaque indicators attached to the mucosa by endoscopy. The interdistance of the indicators reflect axial movements and are documented using video-fluoroscopy. This is a technique not suitable for routine examinations as it is not well tolerated by all patients and involves radiation. Another disadvantage is that each examination has to be subjected to a complicated data-analysis.

Even though there are various methods for assessing the longitudinal contraction of the esophagus, there is still a growing need for improved methods and systems with simple execution procedure, by means of which the motility of tubular anatomical organs such as the esophagus can be studied. In this way, it would be possible to provide knowledge about the relationship between esophageal symptoms and longitudinal contractions of the esophagus.

Circular contractions of the esophagus and LES relaxations that occur e.g. following swallowing, can easily be monitored using for example multiple manometry. A flexible tube is then introduced into the esophageal lumen. Manometric sensors are located at fixed positions along the tube and the pressures are recorded and displayed over time. It should be noted that oscillations of intraluminal pressure in the esophagus as well as in the rest of the gut occur as a function of circumferential contractions. Fixed-position manometry therefore reflects activity in the circular muscular layer. It is also important to note that contractions in the outer longitudinal muscular layer resulting in axial movements do not create intraluminal pressure alterations and can thus not be assessed by conventional manometry.

### SUMMARY OF THE INVENTION

An object of the present invention is to provide an arrangement and a method for an improved assessment of esophageal axial movements.

This object is accomplished by means of an arrangement of the type as mentioned initially, which is further characterized in that it comprises an electrode arrangement arranged along the longitudinal extension of said catheter, a detection unit for measuring the electric potential at least partly along the length of said organ and an evaluation unit for detecting a generally step-like change in the measured electric potential and determining the distance between a position along said catheter corresponding to said step-like change and a predetermined fixed point to which the catheter can be attached.

Said object is also accomplished by means of a method of the type as mentioned initially, which is further characterized in that it comprises assessing the motility by means of an electrode arrangement arranged along the longitudinal extension of said catheter, measuring the electric potential at least partly along the length of said organ, detecting a generally step-like change in the measured electric potential, and determining the distance between a position along said catheter corresponding to said step-like change and a predetermined fixed point to which the catheter can be attached.

By means of the invention, certain advantages are obtained. Firstly, it should be noted that the invention allows in-vivo assessment in realtime of the axial movements of an anatomical organ such as the esophagus, stomach and/or duodenum. In particular, the invention describes a device that monitors longitudinal movements, i.e. contraction and elongation, of the esophagus by use of the electrophysiological characteristics of the esophageal and stomach epithelia.

The invention can be combined with other methods, e.g. conventional manometry, for assessing circumferential contractions. Such a combined arrangement would allow for a combined assessment and on-line display of both circumferential and longitudinal motor actions, i.e. a simultaneous judgement of activity in each of the two muscular layers. Such a combined assessment will exhibit novel insights into the complex basis for symptom generation following GERD, NERD and other gastroesophageal disorders.

In summary, the invention offers a means and method for providing a novel picture of interacting functional properties that can be useful for analyses of the basis for heart burn and subsequent pharmacological developments.

Also, the invention can be useful for diagnosis of hiatus hernia, and also for diagnosis of spread of columnar epithelium into the esophageal body (Barrett's esophagus).

The assessment of esophagogastric movements according to the invention can be further improved by an alternative embodiment of the invention, in which the catheter is additionally provided with a sensor arrangement for indicating the geometrical configuration of said catheter, and which allows assessing the degree of contraction of an anatomical organ based on an analysis of the geometrical configuration.

According to a still further embodiment, the assessment according to the invention can be further improved by using a catheter which is adapted for measurements to be carried out in two different transition zones, suitably in a first transition zone between the esophagus and the stomach, and also in a second transition zone between the stomach and the duodenum. However, occasional bending of the catheter may influence such measurements. By simultaneous recording the geometrical configuration of the catheter, the precision of said measurements can be improved considerably.

Consequently, the general object of the invention, to provide an improved assessment of esophageal axial movements, may include corresponding phenomena in stomach and duodenum that could cause or permit symptom generation.

### DESCRIPTION OF THE DRAWINGS

The invention will now be described in greater detail with reference to certain embodiments and the appended drawings, in which:

FIG. 1 shows in a schematical manner a part of the human esophagus and stomach;

FIG. 2 shows in a schematically manner, and enlarged as compared with FIG. 1, a section of an esophagus in which the present invention is utilized;

FIG. 3 shows a section of a catheter according to a first embodiment of the invention, in a view which is enlarged as compared with FIG. 2; and

FIG. 4 is a diagram showing the measured electric potential in the esophagus and stomach;

FIG. 5 shows an arrangement according to an alternative embodiment of the invention;

FIG. 6a shows a part of the human gastrointestinal system in a first, relaxed, condition;

FIG. 6b shows a part of the human gastro-intestinal system in a second, contracted, condition;

FIG. 7 shows a measurement system in accordance with an alternative embodiment of the invention;

FIG. 8a shows a curvature of a catheter according to the invention in a first condition of the gastrointestinal system; and

FIG. 8b shows a curvature of a catheter according to the invention in a second condition of the gastrointestinal system;

FIG. 9 shows the principles behind a further embodiment of the invention; and

FIG. 10 generally corresponds to FIG. 9 and shows said further embodiment.

### PREFERRED EMBODIMENTS

The present invention relates to an arrangement and a method for assessing the motility of a generally tubular anatomical organ. In particular, the invention can be used for detecting longitudinal movements in the form of muscular contractions in the human esophagus. Such movements can be in the form of inherent contractions of the esophagus, or in the form of a relaxation of the esophagus due to external, distending forces resulting from gastric contraction.

However, the invention is not limited to the human esophagus, but can also be applied in similar applications related to certain types of animals (for example pigs, dogs, cats etc.) having a anatomical structure of the esophagus and stomach which generally corresponds to the description below.

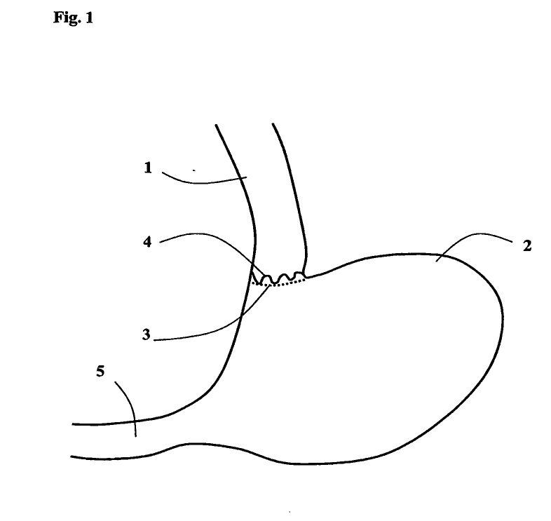

The principles of the present invention will now be described firstly with reference to a preferred embodiment and the appended FIG. 1, which shows in a schematical and simplified manner a human esophagus 1 which extends between the oral cavity (not shown) and the stomach 2.

The esophagus 1 has an inner lining structure (which is not shown in detail in the drawings) which is covered by a mucosa with a squamous epithelium. The stomach 2, on the other hand, has a structure which is characterized by a columnar type of glandular gastric mucosa. The fact that the structure of the lining of the esophagus 1 differs from that of the stomach 2 is used in accordance with the invention when carrying out certain electric potential measurements, as will be described in detail below.

Furthermore, the esophagus 1 and the stomach 2 can be said to be separated from each other at a defined transition zone 3, which is indicated by means of a broken line in FIG. 1. Consequently, the transition zone 3 corresponds to a boundary, or junction, between the esophageal and gastric mucosa in the esophagus 1 and the stomach 2, respectively. Furthermore, it is known that this transition zone 3 is located in close vicinity to the LES (“lower esophageal sphincter”) 4, which has a valvular function and which connects the esophagus 1 with the stomach 2 and which serves to prevent gastric contents from entering into the esophagus 1 while allowing a selective evacuation of swallowed air.

As indicated in FIG. 1, the stomach 2 is also connected to the duodenum 5 and further to the remaining parts of the gastrointestinal system.

It is well known that active ion transport and the inborn electrical resistance of viable epithelia will create an electrical potential difference across a mucosal lining in the esophagus 1 and the stomach 2, respectively. It is also well known that the transmucosal potential difference of esophageal and gastric surface epithelia differs markedly; typically being 12-14 mV in the former and 30-70 mV in the latter. Furthermore, these different electrical properties are directly related to mucosal morphology at the transition zone 3 between esophagus 1 and the stomach 2. The squamous epithelium of the esophagus 1 changes to the columnar type of glandular gastric mucosa at the transition zone 3, which is a very distinct macroscopic location (also referred to as the “Z-line”) which is located in association to the LES 4.

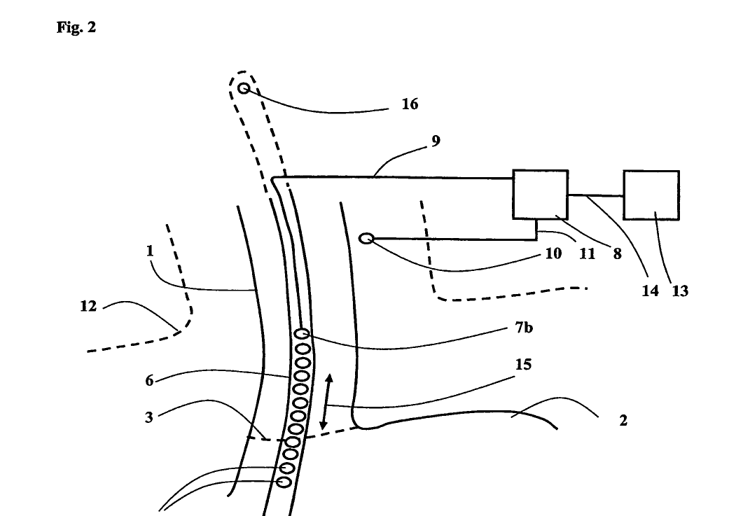

The present invention is based on the principle that the position of the transition zone 3 can be detected by means of electric potential measurements along a certain section of the esophagus 1 and a certain section of the stomach 2. In particular, the invention is based on the fact that potential measurements are carried out on both sides of the transition zone 3. This means that axial movements of the esophagus 1, which are normally caused by muscular contractions of the esophagus 1, can be assessed by continuously detecting and following the actual position of the transition line 3. This principle will now be described in detail with reference to FIG. 2, which shows a view of the esophagus 1, slightly enlarged as compared with FIG. 1.

As shown in FIG. 2, the invention is based on the use of a catheter 6 which is designed and adapted in a manner so as to be introduced into the esophagus 1. More precisely, the catheter 6 is manufactured from a flexible and electrically insulating (plastic or rubber) material having a certain rigidity. The rigidity of the material for the catheter 6 is preferably chosen so that it is sufficiently stiff for introducing the catheter 6 into the esophagus, while still assuming its intended form even if the esophagus should contract in its longitudinal direction.

FIG. 2 discloses in a schematical manner that the catheter 6 comprises a series of electrodes 7. According to a first embodiment of the invention, said electrodes 7 are constituted by a plurality of lumens extending along the catheter 6 and arranged with openings which are arranged in a spaced apart manner in the longitudinal direction of the catheter 6. According to a second embodiment of the invention, said electrodes 7 are constituted by a series of electrically conductive contacts, preferably by a suitable metal, which are attached to the outer surface of the catheter 6 at predetermined interdistances, preferable between 0.5 and 1 cm.

In the case of the first embodiment, each lumen is connected to a unit 8 for feeding a suitable electrolyte through each lumen and for measuring the electrical potential at positions along the esophagus which correspond to the positions of the openings of each lumen. In case of the second embodiment, each contact is connected to a unit 8 for measuring the electrical potential at positions along the esophagus which correspond to the positions of the contacts.

Irrespective of which embodiment of the invention is used, it can be noted that FIG. 2 indicates the detection unit 8 and a connection 9 from the catheter 6 to the detection unit 8. This means that the connection 9 can either be a multi-lumen connector which connects each of the electrodes to the detection unit 8, or a set of electrical cables from each of the electrode contacts to the detection unit 8.

During use, the catheter 6 is preferably introduced via the nose (not shown) and is introduced in a manner so that it extends along the esophagus 1 and reaches a position straddling the gastroesophageal junction, i.e. the above-mentioned transition zone 3, with some of the said electrodes 7 being positioned inside the stomach 2 and the rest inside the esophagus 1. Also, as indicated schematically in FIG. 2, each of the electrodes 7 is connected to a detection unit 8 via a connection 9.

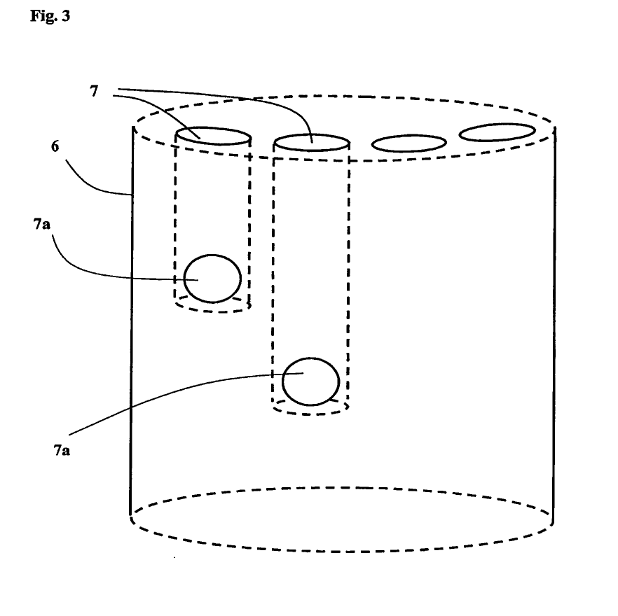

The catheter 6 according to the first embodiment of the invention will now be described in greater detail with reference to FIG. 3, which shows a section of a catheter 6 according to the first embodiment, in a view which is enlarged as compared with FIG. 2. As mentioned previously, the catheter 6 according to the first embodiment comprises a plurality of electrodes in the form of lumens 7, typically in the magnitude of 8-22 and being arranged around the circumference of the catheter 6. However, for reasons of simplicity, only a few of these lumens 7 are shown in FIG. 3. It should be noted that the invention is not limited to any specific number of lumens 7. The particular choice of the number of lumens in each application is primarily determined from the desired accuracy during measurements, also considering other factors such as the cost and complexity of the catheter and the other pieces of equipment used for the measurements. In normal applications, the esophagus 1 can be expected to contract in a manner so that the position of the transition zone 3 moves approximately 2-5 cm. In order to detect the transition zone 3 in an accurate manner, it can be assumed that the distance between the lumen openings should be approximately 0.5 cm. Also, since the set of electrodes must straddle the transition zone 3, it is suitable with 8-22 electrodes.

Each lumen 7 is formed as a tubular duct extending along the catheter 6 and ending with an opening 7a at a given position along the catheter 6. In particular, the openings 7a corresponding to all the lumens 7 are spaced apart along the catheter 6, preferably in a generally even manner along the longitudinal extension of the catheter 6. This means that the openings 7a face the interior of the esophagus 1 at a plurality of positions at generally even distance along its extension.

Consequently, according to the first embodiment, the catheter 6 is constituted by a plastic tube provided with electrodes 7 in the form of multiple channels, each ending in a sidehole 7a on a fixed position along the catheter 6. An electrolyte (preferably physiological saline; ie. 150 mM NaCl) is flowing at a constant and slow rate through each side hole 7a into the intestinal lumen and act as conductor to an external volt-meter. Each channel thus acts as an separate electrode 7.

It should be noted that FIG. 3 is schematical and that in the case of this first embodiment, the openings 7a will be distributed around the outer periphery of the catheter 6.

Consequently, the electric potential difference between each of the electrodes 7 of the catheter 6 and a further electrode 10, which is used as a reference, can be measured by means of the detection unit 8. According to the embodiment, the reference electrode 10 is preferably arranged subcutaneously in the individual on which measurements are made, or in the bloodstream via an electrolyte bridge. Furthermore, the reference electrode 10 is connected to the detection unit 8 via a further connection 11.

With reference to FIG. 2, the individual who is the subject of the measurements is indicated with broken lines and reference numeral 12. Also, in order to measure said potential difference between the electrodes 7 of the catheter 6 and the reference electrode 10, the detection unit 8 is provided with a voltage measurement unit which is preferably in the form of a conventional high impedance voltmeter. The detection unit 8 is furthermore connected to an evaluation unit 13 via a further connection 14. The evaluation unit 13 is preferably computer-based and is adapted for assessing the amount of longitudinal movement of the esophagus 1 based on the electric potential measurements as provided by the detection unit 8. Also, the evaluation unit 13 can be associated with a display (not shown) for indicating graphically the results of the assessments of the movement. In general terms, the evaluation unit is arranged for detecting at least one generally step-like change in the measured electric potential and determining the distance between positions along said catheter corresponding to said step-like change and a predetermined fixed point (to be explained below) to which the catheter can be attached.

According to the invention, values indicating the potential difference between each of the catheter's electrodes 7 and the reference electrode 10, i.e. the potential difference along a section straddling the transition zone 3, are measured by means of the detection unit 8 and are forwarded to the evaluation unit 13. In this regard, the potential difference can be expressed as a function V(d) in which the potential difference V is a function of the distance d from the nostril of each of the catheter's electrodes 7, i.e. the potential difference can be denoted as V(d). This requires the catheter 6 to be fixed at a certain point in the nose, i.e. so that it does not move in the longitudinal direction. Alternatively, the upper end portion of the catheter 6 can be fixed in some other suitable reference point, for example in the oral cavity or throat. In FIG. 2, the fixed point (i.e. preferably the nose) is schematically indicated by means of reference numeral 16.

Since the potential difference is measured on both sides of the transition zone 3, the catheter's electrodes 7 must be arranged so that, during measurements, they straddle the transition zone 3. This means that the position of the transition zone 3 can be detected as a generally step-like change in electric potential at some position along the catheter 6 even if the esophagus 1 should contract or become elongated, i.e. as indicated by means of an arrow 15 in FIG. 2.

As an alternative to the embodiment shown in FIG. 2, in which the reference electrode 10 is constituted by a subcutaneously arranged reference electrode, the reference electrode can also be in the form of one of the existing electrodes, for example the uppermost one of the electrodes 7, as indicated by means of reference numeral 7b in FIG. 2. Consequently, the first embodiment relies on a measurement of the transmucusal potential difference, whereas the second embodiment is not measured in a transmucosal manner but instead relies on the relative potential difference between each of the electrodes as compared with the particular electrode which is chosen as a reference.

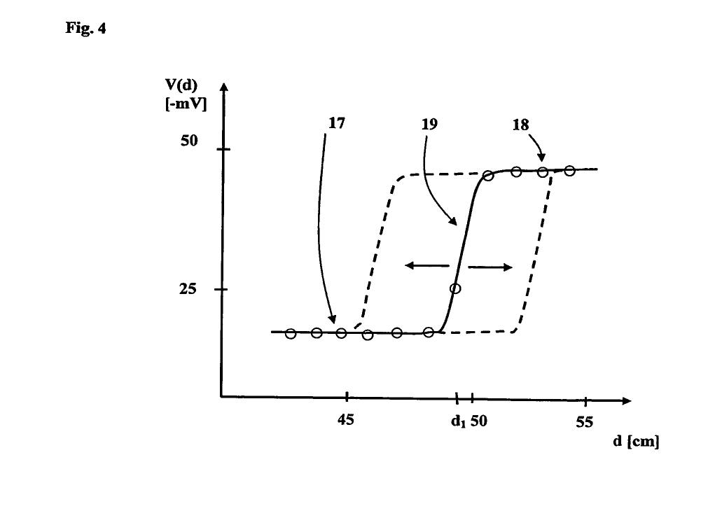

In FIG. 4, the electrical potential differences V(d) between each electrode and the reference electrode 10 are displayed as a function of the distance d from the nostril. This means that a plurality of potential measurements are carried out at positions along the esophagus which correspond to the positions of the catheter's electrodes 7. These positions are indicated by means of small circles in FIG. 4.

Due to the fact that there is a sharp and distinct step in potential along the passage from the esophagus to the stomach (or vice versa), as explained above, the transition zone 3 will be identified as the position where the potential difference is making a step from esophageal values to gastric ones (or vice versa). This means that a first section of the curve in FIG. 4, indicated by means of reference numeral 17, corresponds to generally equal potential values in the esophagus, whereas a section 18 of the curve corresponds to generally equal potential values in the stomach. Finally, the transition zone is indicated as a third, steep section 19 in the curve which forms the step-like change. In this regard, the term “step-like change” is used in this context to describe the fact that the potential values along the catheter rather suddenly changes from relatively equal values to substantially different (i.e. higher or lower) values, thereby forming a relatively steep slope in the plot of the potential as a function of the distance from the fixed point 16.

Furthermore, in FIG. 4 a contraction (shortening) is indicated by means of an arrow pointing to the left, and a relaxation (elongation) is indicated by means of an arrow pointing to the right.

The invention can be used for detecting contractions and extensions due to muscular activity in the longitudinal direction of the esophagus. This is indicated with a broken line in FIG. 4, which corresponds to a contraction of the esophagus, and a displacement of the transition zone in a direction upwards, i.e. towards the oral cavity. In other words, the distance from the fixed point to the transition zone, which is indicated as d₁ in FIG. 4, will decrease as a result of the contraction, i.e. the shortening of the esophagus. This means that the invention is used for assessing the motility of the esophagus 1 with reference to the catheter 6, which in turn is adapted to be attached to a predetermined fixed point 16 during assessment of the motility.

For presentation by means of the evaluation unit 13 (see FIG. 2), the data are preferably displayed in the following three dimensions: distance on the x-axis, the potential difference (alternatively, as a delta value displayed at each distance; for example the difference between one proximal esophageal electrode and the rest of the esophageal and stomach electrodes) on the y-axis; and time on the z-axis.

According to a suitable embodiment of the invention, it can be used so as to provide a simultaneous measuring of the potential difference across the esophagogastric mucosa and measuring of pressure at the same position. It is known that pressure in the flow line can simultaneously be measured on the outside of the body and be displayed separately and used for manometric analyses. Pressure alterations in this low-compliance flow system are thus dependent on changes in flow resistance which in turn will depend on the hydrostatic pressure that is present in the esophageal lumen due to circular muscular activity.

The principles for manometric measurements are previously known per se. However, it can be noted that a combined assessment of both circumferential and longitudinal contractions of the esophagus can be expected to provide new insights into disorders of the esophagus and stomach.

An alternative embodiment is that the catheter consists of materials with low electrical resistance that are isolated into several conductors in a solid state manner, also integrating other sensor systems for example used for manometry, ultrasound and impedance measurement.

Furthermore, the potential measurements can be combined with further types of measurements, such as ph-metry or impedance measurements along the catheter. In this way, the subsequent analysis can be more accurate. Also, time savings can be made due to the fact that several parameters are detected at the same time.

Multiple channel manometry allow for identification recording of dynamics of the high-pressure zone in the LES. Furthermore, by analysing the breathing dependent pressure oscillations superimposed on intraluminal pressures it is possible to identify with very high accuracy the functional border between the thoracic (negative inspiration pressure) and abdominal cavities (positive inspiration pressure). This may be of particular interest in cases with hiatus hernia in which the high pressure zone of the LES may be distorted by the surrounding negative pressure in the thorax. This picture is combined with a position statement on the Z-line by use of multiple potential difference recordings, is the basis for a novel diagnostic tool for hiatus hernia, particularly those of the sliding type that can be difficult to catch during barium swallow or endoscopic investigations.

The characteristic potential difference of columnar gastrointestinal mucosal epithelium can also be used for diagnosis of pathological occurrence of such epithelium within the esophageal lumen. This condition is termed Barrett's esophagus and is considered to be a prominent risk for later development of cancer of the esophagus.

Preferably, the invention should be combined with other assessments of gut motor activity, e.g. manometry, recordings of flow of luminal contents (e.g by impedance measurements) or other functional assessments (with e.g. ultrasound).

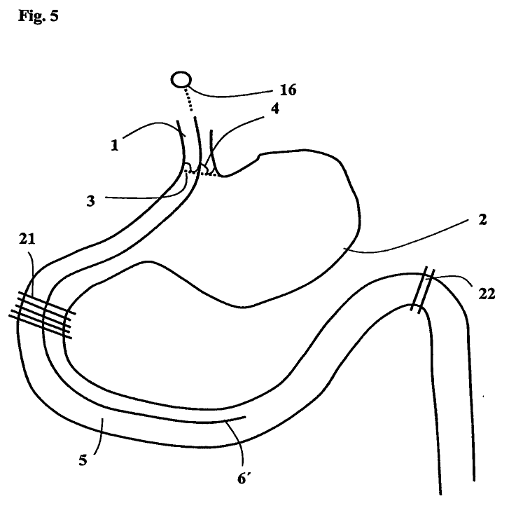

As symptom generation depend on either powerful endogenous contractions or distension/pulling of the longitudinal musculature, both resulting in axial movements of the organ, it is of great value to assess mechanical events in neighbouring organs, i.e. stomach and duodenum. The assessment of contractions and extensions of the esophagus according to the invention, as explained above, can thus be further improved by means of an alternative embodiment, which is shown in FIG. 5. According to this embodiment, the catheter 6 is provided with a sensor arrangement for indicating the geometrical configuration of said catheter 6′, as will be described in detail below.

The duodenum 5 is a generally tubular organ in the gastrointestinal system of a human or certain animals. The gastrointestinal tract is principally a two-layer muscular tube consisting of one inner layer with the muscular fibres oriented in the circumferential direction and one outer layer with muscular fibres oriented in the axial direction. The mechanical functions due to activity in these muscular layers differ somewhat along the gut; the first part; i.e. the esophagus, being primarily transporting; the stomach acts as a reservoir and has a grinding function as well as transporting properties. The latter function is subjected to precise regulation in relation to the digestive capacity in order to deliver matched portions of chyme from the stomach and into the small intestine.

It is well known that contractions of the circular muscular layer (i.e. consisting of the above-mentioned muscular fibres oriented in the circumferential direction) of the generally tubular organs in the gastrointestinal system create pressure gradients that mix and propel the luminal contents. Looked upon from a functional point of view, the circular muscular layer acts as a peristaltic pump. The function of the longitudinal muscular layer (i.e. consisting of the above-mentioned muscular fibres oriented in the axial direction) of the gastrointestinal system, on the other hand, is less obvious and has because of lack of appropriate methodology not been as extensively studied as the circumferential activity. An axial shortening of the gut may be considered to contribute to propulsion by creating components of a piston pump where the gut wall acts as a moving cylinder and the luminal contents acts as a passive piston. Integrated with circular peristaltic activity, such mechanical events may create complex and effective forces for propulsion and mixing.

As indicated schematically in FIG. 5, the duodenum 5 can be said to be fixed in a point, or rather an area 21. The fixing area 21 for the duodenum 5 is defined due to the fact that a descending part of the duodenum 5 (being formed as a continuation of the stomach 2) is fixed retroperitoneally, thereby forming a first anatomically fixed area indicated by means of reference numeral 21 in FIG. 5. Furthermore, a second anatomically fixed area 22 is defined due to the fact that the duodenum 5 is suspended by means of the so-called Treitz ligament. This area is indicated by means of reference numeral 22 in FIG. 5.

According to this alternative embodiment, the assessment of the motility of the esophagus can be improved by also providing an assessment of the geometrical configuration of the duodenum. Said alternative embodiment is shown in FIG. 5 and comprises a catheter 6′ which can be said to be constituted by two sections, i.e. a first section extending generally through the esophagus 1, the stomach 2 and into the duodenum 5, and a second section further extending along the duodenum 5 (at least from a point before the first fixing area 21).

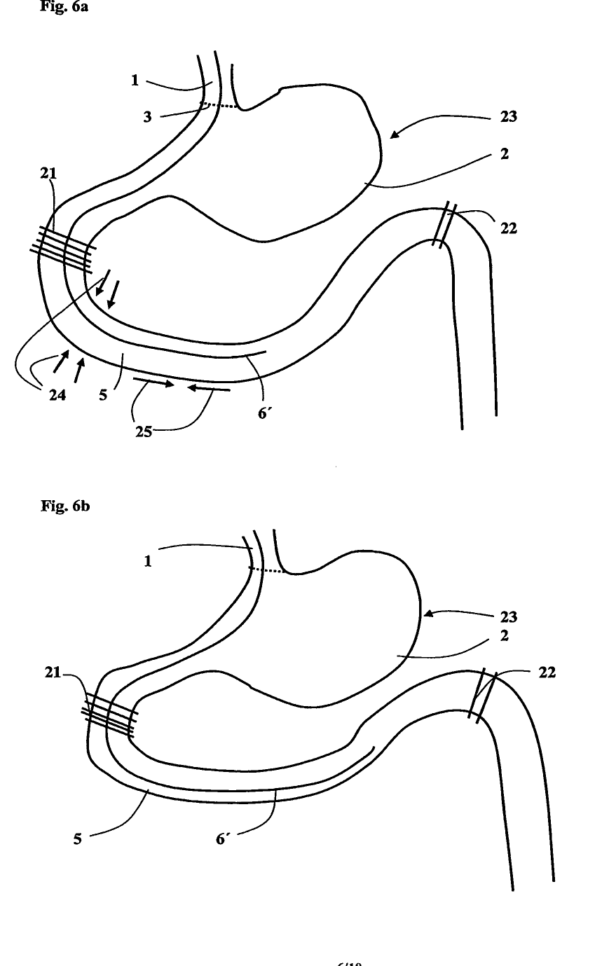

FIGS. 6a and 6b are schematical and simplified drawings of a part of the gastrointestinal system 23 of a human, in two different conditions. FIG. 6a shows a part of the upper gastrointestinal system of a human and in a relaxed condition, whereas FIG. 6b shows the same part of the upper gastrointestinal system in a contracted condition. As indicated in FIG. 6a, the duodenum 5 extends in a curve-like manner from the lower section of the stomach 2.

As indicated in FIG. 6a by means of a first set of arrows 24, the duodenum 5 functions so as to contract in a circumferential direction. For this reason, the first set of arrows 24 are arranged in a direction which is generally transversal to the extension of the duodenum 5. Such circular contractions result in pressure gradients in the duodenum 5 that mix and propel the luminal contents. Furthermore, as indicated in FIG. 6a by means of a second set of arrows 25, the duodenum 5 also functions so as to contract in a generally longitudinal direction along the duodenum 5. This axial shortening of the duodenum, which is due to activity in longitudinally extending muscular fibres, can be assumed to contribute to the propulsion of the luminal contents.

FIG. 6a indicates the duodenum 5 in a first condition which is a relaxed condition, i.e. a relaxed condition of the longitudinally extending muscular layers in the duodenum 5. In contrast to this first condition, FIG. 6b indicates in a schematical manner the duodenum 5 in a second condition, more precisely a contracted condition. The contracted condition occurs as a result of an axial shortening of the duodenum 5 due to contraction of said longitudinally oriented muscular layer.

According to this alternative embodiment, measurements of the degree of such axial, or longitudinal, contractions of the gastrointestinal system, for example in the duodenum 5 as shown in FIG. 6b, can provide valuable information which can be used together with the above-mentioned detection of the transition zone 3 by means of electric potential measurements along a certain section of the esophagus 1 and a certain section of the stomach 2. To this end, the alternative embodiment is based on the use of the catheter 6′ which extends through the esophagus 1, the stomach 2 and duodenum 5. The catheter 6 is manufactured from a material having a low rigidity and which is capable of being introduced into the gastrointestinal system. Preferably, a plastic or rubber material is used.

The catheter 6′ will assume a different configuration during the relaxed state (FIG. 6a) as compared with its configuration during the contracted state (FIG. 6b). Furthermore, according to the embodiment, the physical shape, i.e. the geometrical configuration of the catheter 6′, will be determined during the relaxed and contracted state, respectively. Information related to the configuration of the catheter 6′ will then be used in assessing the degree of contraction of the duodenum 5.

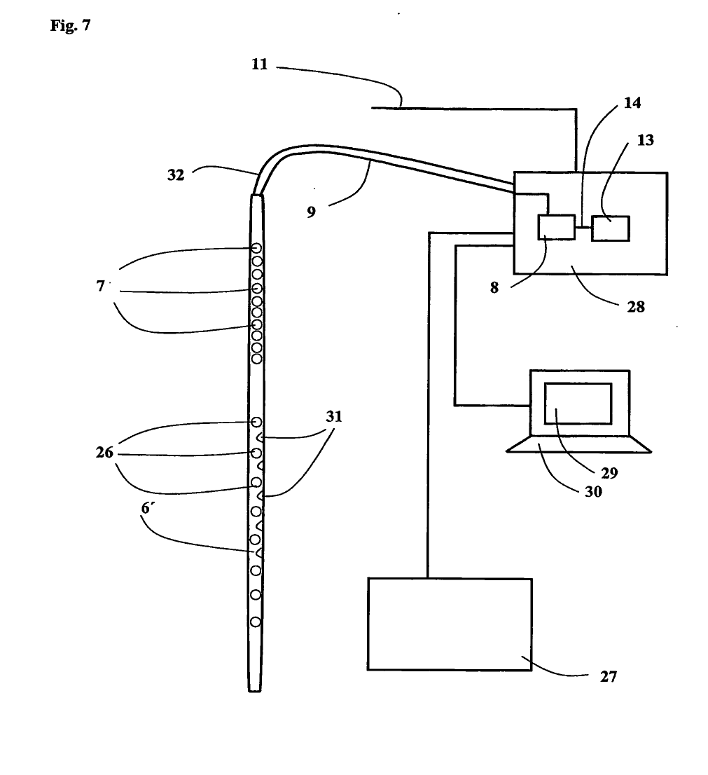

This principle will now be described in greater detail. With reference to FIG. 7, an arrangement according to the embodiment is shown, i.e. with a catheter 6′ having a number of electrodes 7 arranged to straddle the transition zone 3 as described above. In FIG. 7, the catheter 6′ is shown in a condition in which it is not introduced into a gastrointestinal system. Furthermore, the catheter 6′ comprises a sensor arrangement adapted for capturing the geometrical configuration of the catheter 6′ inside the duodenum 5. Said sensor arrangement is preferably constituted by a plurality of radiologically opaque markers 26 which are positioned along a lower portion of the catheter 6′, i.e. below the sensors 7. The markers 26 are adapted so as to be observable, i.e. visualized, by means of fluoroscopy, which is a technique for obtaining x-ray images of a human or an animal. According to this embodiment, the catheter 6 with its markers 26 are used in connection with a fluoroscopy scanning device 27 which is adapted for detecting the position of each of said markers 26. The fluoroscopy scanning device 27 forms part of a measuring unit which also comprises a central control unit 28, which preferably is computer-based but which can also be implemented in other ways.

During use of the catheter 6′, the markers 26 will be detectable by means of the x-ray device 27. In particular, the position of each of the markers 26, for example with reference to an orthogonal coordinate system, will be determined. Data related to the positions of each of the markers 26 will be transferred to the control unit 28 for further evaluation. Based on information from the x-ray device 27, the control unit 28 is adapted for determining the degree of longitudinal contraction of the duodenum 5 based on values indicating the geometrical configuration as provided by the markers 26. In other words, the control unit 28 is adapted for assessing the geometrical changes in the configuration of the catheter 6 when the gastrointestinal system goes from a relaxed to a contracted condition.

Furthermore, information regarding the configuration of the catheter 6 can be displayed on a display 29 forming part of a computer 30. This means that the control unit 28 and computer 30 with its display 29 and associated software together form part of a measuring unit which can be used for analysis of altered catheter-curvatures including estimation of changes in the axial direction of the gut wall. Such an analysis can be used for improving the analysis of the movements of the above-mentioned transition zone 3. To this end, the catheter 6 according to FIG. 7 has both the radiologically opaque markers 26 positioned along the catheter 6′ and also electrodes 7 arranged to straddle the transition zone 3 as described above. In this manner, the catheter 6′ can be said to be divided into two sections as described above.

Also, the catheter 6′ is preferably provided with means for manometry measurements by means of a number of side-holes 31. The manometric sensors are connected to the control unit 28 by means of a connection 32, whereas the electrodes 7 are connected to the unit 8 for feeding electrolyte and for measuring the electrical potential at positions along the esophagus. Said unit 8, its connection 9 to the catheter 6′, and also the evaluation unit 13 and its connection 14, are shown schematically in FIG. 7. The connection 11 from the reference electrode (not shown in FIG. 7) is also shown schematically in FIG. 7.

Consequently, the catheter arrangement according to the embodiment shown in FIGS. 5-7 forms an integrated arrangement both for detecting motility of certain parts of the gut, for example the duodenum or the colon, and also for assessing the motility of the esophagus. These two measurements can be carried out simultaneously by means of the arrangement according to FIG. 7. This is an advantage from a practical point of view, since it reduces the time, cost and effort for such measurements. Also, the results from the two measurements can be correlated so as to provide new insights as to certain disorders of the esophagus, stomach and duodenum.

The invention can be used to provide a simultaneous measuring pressures at the same position. It is known that pressure in the flow line can simultaneously be measured on the outside of the body and be displayed separately and used for manometric analyses. Pressure alterations in this low-compliance flow system are thus dependent on changes in flow resistance which in turn will depend on the hydrostatic pressure that is present in the lumen due to circular muscular activity.

The principles for manometric measurements are previously known per se. However, it can be noted that a combined assessment of both circumferential and longitudinal motility can be expected to provide new insights into disorders of the esophagus, stomach and duodenum.

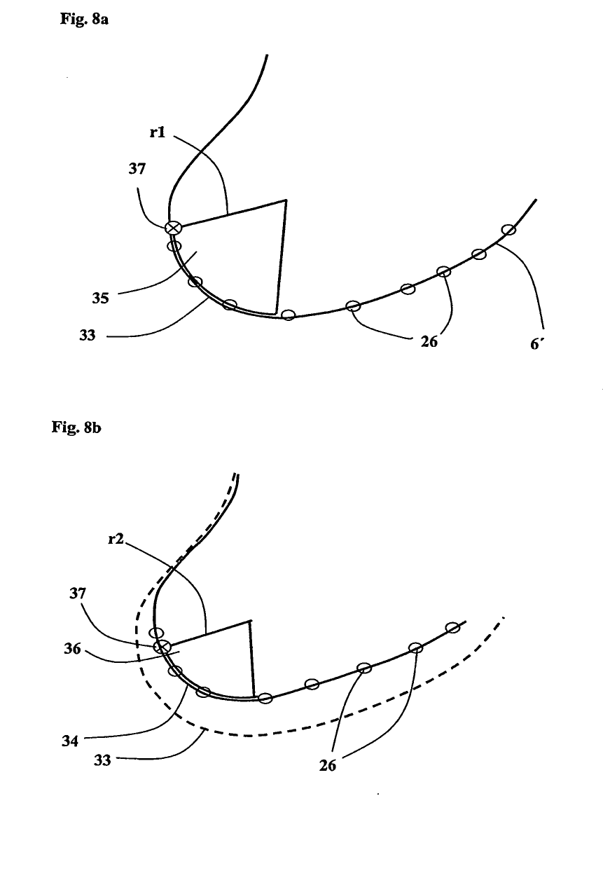

The principles behind the alternative embodiment will now be described in further detail with reference to FIGS. 8a and 8b. FIG. 8a shows in a simplified and schematical form a first curvature 33 of the catheter, as represented by the plurality of markers 26 disposed along the length of the catheter 6′. Furthermore, the curvature shown in FIG. 8a corresponds to the relaxed state of the duodenum (cf. FIG. 6a).

On the other hand, FIG. 8b shows a second curvature 34 of the catheter which corresponds to a contracted state of the duodenum (cf. FIG. 6b). For clarity, the above-mentioned first curvature 33 is also shown in FIG. 8b, as a reference. In the contracted state of the duodenum, the curvature of the catheter will be more sharp. According to the alternative embodiment, a measure of the degree of contraction is obtained by choosing suitable geometrical parameters which define the amount of contraction of the catheter and consequently also of the duodenum.

During longitudinal relaxation a mobile segment of the duodenum 5 tends to form a curve starting at the anatomically fixed area 21. When an axial shortening occurs due to contraction of the longitudinally oriented muscular layer, this curvature will become more pronounced, as shown in FIG. 8b. It follows that the shape of the intraluminally situated flexible catheter will attain and follow changes in the luminal extension.

Recording of the shape of the catheter makes it possible to calculate the radius of a hypothetical circle which fit to the curvature under study. The proportionality of the radius to the circumference is useful as an absolute measure of longitudinal movements in relation to the fixed part of duodenum 5.

As indicated in FIG. 8a, a circular segment 35 representing the curvature of the catheter 6′ is fitted into the curvature 33 representing the relaxed condition of the duodenum 5. In a similar manner, as indicated in FIG. 8b, a further circular segment 36 representing the curvature of the catheter in the contracted condition is fitted into the curvature 33. Furthermore, in FIGS. 8a and 3b, the circular segments 35, 36 extend from a fixed reference point 37 which is the same in both the relaxed and the contracted state of the duodenum, and which generally correspond to the above-mentioned first fixing area 21.

Preferably, the invention is arranged so that the circular segments 35, 36 are used so as to calculate an imaginary circumference for both the relaxed and the contracted condition of the duodenum. Due to the fact that the radius r1 of the relaxed state (FIG. 8a) and the radius r2 of the contracted state (FIG. 8b) are indicated, values of the corresponding circumferences can be calculated using the well-known formula 2*π*r. The calculated values of the two circumferences are then used to assess the degree of longitudinal contraction of the duodenum.

Additionally, the geometrical configuration of the catheter 6 can be displayed on the display 29 cooperating with the computer 30, which in turn is connected to the control unit 28.

Alternatively, more than one curvature can be defined along the extension 33, 34 of the catheter. The principles of this embodiment are not shown in the drawings. For example, a first curvature can be defined before the first fixing area 21, a second curvature can be defined immediately after the first fixing area 21 and a third, more flat curvature closer to the second fixing area 22. This will further increase the possibility for even more accurate assessment of the longitudinal contractions of the duodenum over time.

The embodiment is not limited to the above-mentioned method of determining circular segments as indications of the geometrical configuration. Generally, any method for determining the position, inclination, curvature and shape of the catheter 6 along its extension can also be used in order to determine the degree of longitudinal contraction of the duodenum.

According to further embodiments, the above-mentioned plurality of radiologically opaque markers 26 can be replaced with other sensor devices serving generally the same purpose. For example, a sensor arrangement in the form of a plurality of strain gauges can be used for providing measurements of the bending of the catheter, i.e. measurements of the geometrical configuration of the catheter. Strain gauges can be configured in a Wheatstone bridge, which is an electrical network having four resistive elements. One or several of these elements can be constituted by strain gauges. If bending strain acts upon any one of these strain gauges, the resistance of this gauge will change. By means of the Wheatstone bridge configuration, variations in resistance generated by the strain gauges can be measured.

A number of such strain gauges can be arranged along a catheter. Each of the gauges is electrically connected as a part of a Wheatstone bridge, for providing measurements reflecting the bending strain acting upon the catheter during use, and consequently also reflecting the geometry of the catheter.

According to a further alternative, the catheter can be provided with a sensor arrangement in the form of one or more optical fibers, preferably extending along the inside of the catheter so as to follow the curvature of the catheter. Furthermore, it is previously known to provide an optical fiber bend sensor for measuring the degree and orientation of bending of said fiber. The degree of bending can for example be assessed by detecting an interference pattern resulting from light propagating through such a fiber or fibers and being modulated as a result of bending of the fiber. To this end, the fiber can for example comprise bending-sensitive elements which can be positioned in the catheter so as to reflect any bending thereof. In such a manner, the degree and orientation of bending which is present in the fiber (and consequently also in the catheter) can be assessed, and can consequently also be used for providing measurements of the geometrical configuration of the catheter.

According to a further alternative embodiment, the catheter can be provided with a sensor arrangement in the form of a number of transponders which are adapted for providing information related to their positions and for transmitting said information to an external transmitter/receiver unit. To this end, the transmitter/receiver unit is provided with an antenna for communicating with said transponders. Also, the transmitter/receiver unit is connected to a control unit which is adapted to assess the actual geometric configuration of the catheter based on information regarding the positions of each of the transponders, as captured by the transmitter/receiver unit with its antenna.

The transponders can for example be in the form of passive transponder tags which cooperate with the antenna for detecting the position of each such tag. In this manner, such a sensor arrangement can be used for providing measurements of the geometrical configuration of the catheter.

Also, the embodiment can be combined with other assessments of gut motor activity, e.g. manometry or recordings of flow of luminal contents (e.g by impedance measurements) or other functional assessments (e.g. recording of transmucosal potential difference).

The possibility of combining the inventive assessment of longitudinal muscular activity with manometry measurements is indicated schematically in FIG. 7, which shows that the catheter 6 is provided with means for manometry measurements by means of a number of side-holes 31. In this manner, manometric sensors are located at fixed positions along the tube and the pressures are recorded and displayed over time. To this end, the manometric sensors are connected to the control unit 28 by means of the connection 9. Such a multilumen manometric catheter is used to record changes in intraluminal pressure due to circumferential contraction caused by activity in the gastrointestinal circular muscular layer. The principles for such manometric measurements are previously known per se. For this reason, they are not described in any detail here.

The alternative embodiment can be used to estimate gut wall longitudinal movements by assessing and displaying the three dimensional shape of an intraluminal flexible catheter positioned at certain locations along the intestinal extension.

The assessment of the geometrical configuration can be used for assessing muscular contractions in various parts of the gastrointestinal system, for example the duodenum, but for example also the colon. This assessment is also applicable at certain regions of the gut that can be reached by an intubation procedure and where there exist mobile parts of the gut as well as a part fixed to known anatomical positions. An example of such anatomical fixation points of the gut are the descending part of the duodenum with both the proximal and distal part as mobile parts, as shown in FIGS. 6a and 6b. Another example is the ascending and descending large bowel with the transverse colon being mobile (not shown). A third example is the anorectal association to the pelvic floor with sigmoid colon as mobile part. Also, this assessment can be used in connection with other generally tubular anatomical organs than those of the gastrointestinal system. For example, the invention can be used for measurements in the ducts of the biliary or urogenital tracts.

In summary, the alternative embodiment shown in FIGS. 5-8 is used for assessing axial movements in certain parts of the gut, for example the duodenum, by analysing the geometrical configuration of such parts, combined with a process for assessing the motility of the esophagus 1 with reference to a catheter 6′, as described above, wherein said catheter 6′ is attached to a predetermined fixed point 16.

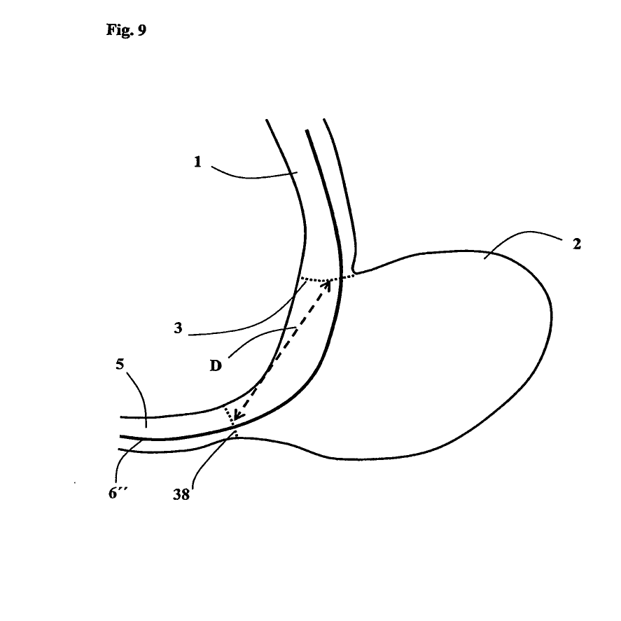

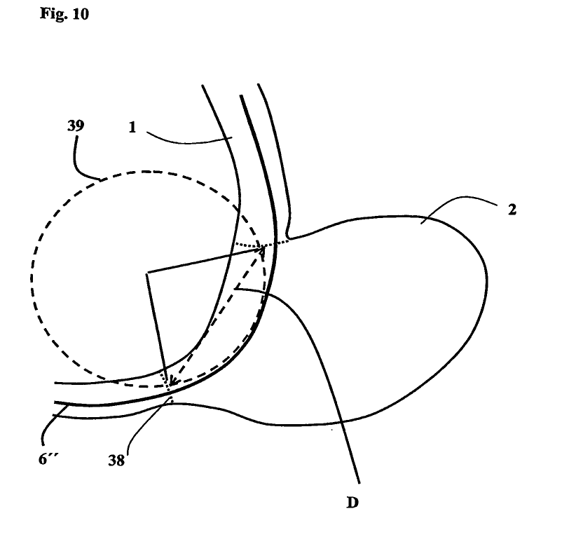

According to a still further embodiment, involving potential difference measurements, such measurements can be carried out both in the transition zone 3 between the esophagus 1 and the stomach, and also in a second transition zone 38 between the stomach and the duodenum 5, where the transmucosal potential difference drops distinctly from typically 30-55 mV in the stomach to 4-6 mV of the duodenum. This principle is shown in FIG. 9, wherein said second transition zone is indicated by means of reference numeral 38. By detecting both these transition zones 3, 38 by means of potential difference measurements as described above, the length of the stomach (i.e. the so called minor curvature of the stomach), including any contractions of the esophagus or stomach, can be measured. In such case, a catheter 6″ should be used with comprises two sets of sensors (corresponding to the sensors 7 shown in FIGS. 2 and 7), said sets of sensors being positioned so as to straddle the two transition zones 3, 38. This embodiment can also be combined with the arrangement for assessing the geometrical configuration as described above with reference to FIGS. 5-8. In particular, the embodiment can be used to compensate for the bending of the catheter 6″ that occur in the stomach lumen and that seriously can the distort the assessment of the linear distance D between the two mucosal transition zones 3, 38. This is done by assessing the geometrical configuration based on the curve-shape defined by the catheter 6″, as shown in FIG. 9, and determining said distance D from the assessment of said geometrical configuration. This is shown schematically in FIG. 10, which indicates that an assessment of the geometrical configuration of the catheter 6″ can be used for determining said distance D. This is carried out in a manner which corresponds to FIGS. 8a and 8b, as described above, wherein a hypothetical circle 39 is fitted to the curvature of the catheter 6″ and wherein the proportionality of the circle's 39 radius to the circumference can be used to calculate the distance D.

The invention is not limited to the above-mentioned embodiment but can be varied within the scope of the appended claims. For example, the invention can be used for measurements related to humans and animals as indicated above.

Finally, the invention can be used in connection with measurements related to other generally tubular anatomical organs than the esophagus, in humans or animals, more precisely organs in which different electrical properties exist in two adjacent sections having different mucosal morphologies and being separated by a transition zone between two such sections.

According to the invention, a peroral or transnasal route of intubation of the catheter 6 may apply. In the case of a peroral intubation, the catheter is preferably fixed in the front teeth of the individual on which measurements are made.

1. Arrangement for assessing the motility of a generally tubular anatomical organ, comprising:
a longitudinally extending catheter being adapted for introducing into said organ and being adapted to be attached to a predetermined fixed point when assessing motility
an electrode arrangement arranged along a longitudinal extension of said catheter,
a detection unit for measuring the electric potential (V(d)) at least partly along a length of said organ, and
an evaluation unit for detecting a generally step-like change in the measured electric potential and determining the distance between a position along said catheter corresponding to said step-like change and the predetermined fixed.

2. The arrangement according to claim 1, wherein the detection unit is to measure said electric potential (V(d)) in organs having different electrical properties due to different mucosal morphology in said organs.

3. The arrangement according claim 1 wherein said electrode arrangement is arranged to straddle a transition zone, thereby measuring the electric potential on both sides of said step-like change to detect a position of the transition zone in relation to said fixed catheter.

4. The arrangement according to claim 1, wherein said electrode arrangement comprises a plurality of electrodes mounted longitudinally spaced apart along said catheter.

5. The arrangement according to claim 4, wherein said electrodes include a plurality of separated lumens extending along the interior of said catheter, each of said lumens having an opening facing the inside of said organ.

6. The arrangement according to claim 4, wherein said electrodes include a plurality of electrically conductive contacts arranged in a spaced-apart configuration along said catheter.

7. The arrangement according to a reference electrode is connected to said detection unit.

8. The arrangement according to claim 7, wherein said reference electrode is adapted for arranging subcutaneously in, or in contact with, an individual for which an assessment of said motility is to be carried out.

9. The arrangement according to claim 7, one of said electrodes also constitutes a reference electrode.

10. The arrangement according to claim 1, wherein the evaluation unit is adapted for determining the degree of contraction of the esophagus of a human or animal.

11. The arrangement according to claim 1, wherein said catheter also comprises means for side-hole manometry in said organ, for assessing circumferential muscular contractions of said organ.

12. The arrangement according to claim 1, wherein said catheter also comprises sensor means of at least one of ph-metry or impedance measurements along said catheter.

13. The arrangement according to claim 1, wherein said catheter is provided with a sensor for indicating the geometrical configuration of said catheter, and said sensor comprises a measuring unit for cooperating with said catheter and being adapted for assessing the degree of contraction of an anatomical organ.

14. The arrangement according to claim 13, said measuring unit is provided with means for determining the degree of longitudinal muscular contraction of said organ based on values indicating said geometrical configuration.

15. The arrangement according to claim 1, wherein the catheter is adapted for detecting two generally step-like changes corresponding to two transition zones along which the catheter is positioned during use.

16. Method for assessing the motility of a generally tubular anatomical organ, comprising:
introducing a longitudinally extending catheter into said organ,
assessing the motility by an electrode arrangement (7) arranged along the longitudinal extension of said catheter with the catheter at a fixed point,
measuring the electric potential (V(d)) at least partly along the length of said organ,
detecting a generally step-like change in the measured electric potential, and
determining the distance between a position along said catheter corresponding to said step-like change and a predetermined fixed point to which the catheter can be attached.

17. The method according to claim 16, further comprises assessing circumferential muscular contractions of said organ.

18. The method according to claim 16 further comprising detecting values related to the geometrical configuration of said catheter by means of a sensor arrangement provided on said catheter, and determining the degree of longitudinal muscular contraction of an anatomical organ based on said values indicating said geometrical configuration.

19. The method according to claim 18, further comprising determining the degree of longitudinal muscular contraction of said organ based on values indicating said geometrical configuration.

20. The method according to claim 16, further comprising detecting two generally step-like changes corresponding to two transition zones along which the catheter is positioned during use.

21.-24. (canceled)
